(19) United States
(12) Patent Application Publication
(10) Pub. No.: US 2006/0039971 A1
(43) Pub. Date: Feb. 23, 2006

(54) Effervescent composition including alternative hormone replacement therapy agent

(76) Inventors: Robert E. Lee, Hudson, WI (US); Mary Aldritt, Excelsior, MN (US)

Correspondence Address: ALLISON JOHNSON, P.A., LAKE CALHOUN EXECUTIVE CENTER, 3033 EXCELSIOR BLVD., SUITE 467, MINNEAPOLIS, MN, 55416, US

(21) Appl. No.: 10/921,698

(22) Filed: Aug. 19, 2004

(51) Int. Cl. A61K 36/28; A61K 36/48; A61K 36/537; A61K 9/46

(52) U.S. Cl. 424/466; 424/746; 424/757; 424/765

(57) ABSTRACT

An effervescent composition including at least 0.5% by weight of an herbal extract selected from the group consisting of red clover extract, black cohosh extract, sage extract and combinations thereof, at least 0.1% by weight of soy isoflavones, at least 0.1 % by weight of N-acetyl-L-cysteine, and an effervescent agent that includes an acid and a base.

### BACKGROUND

The invention relates to formulating effervescent compositions that include herbal agents for relieving of the symptoms associated with menopause.

Symptoms associated with menopause including hot flashes, night sweats and vaginal dryness traditionally have been treated with various synthetic hormone replacement therapy (HRT) agents. The increased risk of side effects from HRT agents including breast cancer, stroke, heart attack and blood clotting has created a demand for alternative treatment options.

Herbal agents such as herbal extracts of some plants have been reported as being capable of reducing the symptoms of menopause and providing useful alternatives to HRT. Dry herbal extracts are a concentrated form of the herb often obtained by mixing the herbal plant with a solvent and evaporating off the solvent to produce a dry residue, which is then ground into a powder. The herbal extracts and other substances that have been studied for their potential use in HRT include black cohosh, red clover, sage, soy isoflavones, vitamin C, and N-acetyl-L-cysteine.

### SUMMARY

The invention features an effervescent composition that includes at least 0.5% by weight of herbal extract selected from the group consisting of red clover extract, black cohosh extract, sage extract, and combinations thereof, at least 0.1% by weight of soy isoflavones, at least 0.1% by weight of N-acetyl-L-cysteine, and effervescent agent includes acid and base. In one embodiment, the composition includes at least 1.0% by weight herbal extract.

In one embodiment the composition further includes ascorbic acid.

In other embodiments the composition further includes flavor agent, color agent, sweetener, or a combination thereof.

In some embodiments the base includes sodium bicarbonate, sodium carbonate, potassium bicarbonate, calcium carbonate or a combination thereof.

In another embodiment the acid includes citric acid, tartaric acid, or a combination thereof.

In one embodiment, the effervescent composition is in the form of an effervescent tablet that further includes binder and lubricant. In some embodiments, the binder includes sorbitol. In one embodiment, the lubricant includes at least one of mineral oil and polyethylene glycol.

In some embodiments, the tablet exhibits a hardness of at least 5 kilopounds and disintegrates in water having a temperature of about 22° C. in less than 3.5 minutes. In other embodiments, the tablet, when stored in an airtight sealed package at 40° C. and 75% relative humidity for 7 days, is free of puffing. In another embodiment, the tablet, when stored in an airtight sealed package at 40° C. and 75% relative humidity for 30 days, is free of puffing.

In another embodiment, the tablet includes from about 20 mg to about 200 mg of the herbal extract. In other embodiments, the tablet includes from about 50 mg to about 100 mg soy isoflavones. In some embodiments, the tablet includes from about 70 mg to about 150 mg N-acetyl-L-cysteine. In one embodiment, the tablet further includes from about 25 mg to about 100 mg ascorbic acid.

In other embodiments, the tablet includes at least 30 mg black cohosh extract, at least 30 mg red clover extract, and at least 20 mg soy isoflavones.

In other embodiments, the effervescent composition includes from about 1% by weight to about 5% by weight of soy isoflavones, from about 1% by weight to about 5% by weight of red clover extract, from about 1% by weight to about 5% by weight of black cohosh extract, from about 1% by weight to about 5% by weight of sage extract, from about 3% by weight to about 10% by weight of N-acetyl-L-cysteine, and an effervescent agent that includes acid and base.

In one embodiment the effervescent composition includes no greater than about 20% by weight of the soy isoflavones and no greater than about 20% by weight of the N-acetyl-L-cysteine.

In another embodiment, the effervescent composition includes at least 30 mg of a first herbal extract selected from the group consisting of red clover extract, black cohosh extract, sage extract, and combinations thereof, at least 30 mg of a second herbal extract selected from the group consisting of red clover extract, black cohosh extract, sage extract, and combinations thereof, the second herbal extract being different from the first herbal extract, and effervescent agent comprising acid and base.

In another aspect, the invention features a method of making an effervescent composition, where the method includes drying an herbal extract, and combining the herbal extract with an effervescent agent.

In other aspects, the invention features a method of making an effervescent tablet that includes drying an herbal extract, combining the herbal extract with an effervescent agent, and forming the effervescent composition into a tablet having a hardness of at least 3 kilopounds.

The invention features an effervescent composition that provides a unique system for delivering multi-symptom relief to women experiencing menopause. The effervescent tablet provides multiple, natural active agents in discreet and controlled quantities, which in turn provides multi-symptom relief to people suffering from menopause. The effervescent tablet can provide a relatively larger dose of the natural active agent than has been provided in existing dosage forms, permits good active agent absorption rates, disintegrates relatively fast in water and is relatively easy to administer.

Other features and advantages will be apparent from the following description of the preferred embodiments and from the claims.

### GLOSSARY

In reference to the invention, these terms have the meanings set forth below:

The term “effervescent composition” refers to a composition that rapidly gives off gas (e.g., carbon dioxide) bubbles when placed in an aqueous liquid.

The term “herbal extract” refers to a concentrated form of an herb, obtained through solvent extraction, and includes a complex mixture of multiple components of the herb. An herbal extract can be obtained by soaking an herb in a solvent (e.g., water, alcohol, or a combination thereof) for a period of time sufficient to allow various components of the herb to enter into the solvent, separating the solvent and the herb, and then removing the solvent (e.g., by drying, evaporation or a combination thereof) thereby leaving a residue. The resulting residue is the extract. The extract may be in the form of a viscous extract, e.g., a soft solid extract, or a dry solid extract. A dry solid extract, which if not already in powdered form, can be ground into coarse granules or a fine powder. A solid extract also can be diluted with alcohol and water to form a fluid extract or a tincture.

### DETAILED DESCRIPTION

The effervescent composition provides a daily dose of multiple alternative hormone replacement therapy agents an in a single tablet or sachet. The effervescent tablet is sufficiently hard such that the tablet maintains its integrity until use, yet exhibits a desirable disintegration profile. The effervescent tablet preferably has a hardness of at least 3 kilopounds (Kp), preferably at least 5 Kp, from about 5 Kp to about 10 Kp, or even from about 5 Kp to about 8 Kp, as measured on a standard hardness tester (e.g., tablet hardness testers available from Dr. Schleuniger Pharmatron, Inc., Germany), and disintegrates in less than 3.5 minutes, less than 3 minutes, from 1.5 minutes to 3 minutes, or even less than 2.7 minutes, when placed in room temperature (about 22° C.) water. The tablet preferably disintegrates in water to form a dispersion, or even a solution.

The effervescent composition includes multiple alternative hormone replacement therapy agents (e.g., herbal extract(s), soy isoflavones, N-acetyl-L-cysteine, and combinations thereof) and an effervescent agent. Suitable herbal extracts include, e.g., red clover extract, black cohosh extract, sage extract, and mixtures thereof.

Suitable forms of herbal extracts include solids (e.g., powder) and liquids. The herbal extract is preferably in the form of a dry powder. Preferably the herbal extracts are subjected to a drying process before being combined with other components of the effervescent composition. Useful drying processes include exposing the herbal extract to a temperature of from at least 40° C. to no greater than 60° C. for from about 12 hours to 36 hours.

Black cohosh (cimicifuga racemosa) has been reported to reduce hot flashes and relieve vaginal dryness. Black cohosh extract contains triterpene glycosides, isoflavones (formononetin) and salicylic acid. The triterpene glycosides are often present in amounts of from 0.5% by weight to 30% by weight, or even from 1% by weight to 20% by weight. Suitable sources of black cohosh extract include the roots and rhizomes of the black cohosh plant. Black cohosh extract is commercially available from a variety of suppliers including, e.g., BI Nutraceuticals (Long Beach, Calif.). Preferably black cohosh is present in the composition in an amount sufficient to temporarily reduce hot flashes. Preferably the effervescent composition includes at least 0.5% by weight, from about 1% by weight to about 20% by weight, or even from about 1% by weight to about 5% by weight black cohosh extract. A single dose tablet preferably includes at least 10 mg at least about 20 mg, at least about 30 mg, no greater than about 100 mg, no greater than about 60 mg, or even no greater than about 50 mg black cohosh extract.

Red clover (trifolium pratense) has been reported to increase blood flow in arteries, and to reduce hot flashes and night sweats. Red clover extract contains isoflavones (genistin, daidzin, biochanin, formononetin). Red clover extract is typically derived from the flowers and leaves of red clover. A useful red clover extract is commercially available from Buckton Scott Nutrition, Inc. (Fairfield, N.J.). Preferably red clover is present in the composition in an amount sufficient to temporarily reduce hot flashes, night sweats or a combination thereof. Preferably the effervescent composition includes at least 0.5% by weight, from about 1% by weight to about 20% by weight, or even from about 1% by weight to about 5% by weight red clover extract. A single dose tablet preferably includes at least 10 mg at least about 20 mg, at least about 30 mg, no greater than about 100 mg, no greater than about 60 mg, or even no greater than about 50 mg red clover extract.

A variety of sages are available including, e.g., aromatic sages, and chia sages. Examples of aromatic sages include salvia apiana, salvia candelabrum, salvia clevelandii, salvia elegans, salvia fulgens, salvia greggii, salvia lyrata, salvia miltiorrhiza, salvia officinalis (e.g., S. o. Purpurascens, S. o. Tricolor, S.o. Berggarten, S.o. Icterina, and S.o. Alba), salvia pratensis, salvia sclarea, and salvia verticillata. The aromatic sages have been reported to strengthen the lungs and provide relief from infection, inflammation, or a combination thereof. Examples of chia sages include, salvia arizonica, salvia carnosa, salvia columbariae, salvia polystachya, and salvia potus.

Sage (e.g., salvia officinalis) has been reported to reduce night sweats. Sage contains volatile oils, diterpenes, triterpenes, flavonoids and phenolic acids. Useful sage extracts are obtained from the sage plant as a whole including leaves. A useful sage extract is available from Buckton Scott Nutrition, Inc. (Fairfield, N.J.). Preferably sage is present in the composition in an amount sufficient to temporarily reduce night sweats. Preferably the composition includes at least 0.5% by weight, from about 1% by weight to about 20% by weight, or even from about 1% by weight to about 5% by weight sage extract. A single dose tablet preferably includes at least 10 mg, at least about 20 mg, at least about 45 mg, no greater than about 80 mg, no greater than about 60 mg, or even no greater than about 40 mg sage extract.

Isoflavones are found in a variety of plants including soybeans, black cohosh and red clover. Isoflavones have a similar structure to estrogen and can act as weak estrogens in the body. Isoflavones are also known as phytoestrogens or plant estrogens.

Soybeans are a major source of isoflavones. Soy isoflavones have been reported to reduce hot flashes and decrease levels of breast cancer. Soy isoflavones are available in compounds in amounts between 10% by weight and 90% by weight, or even from 25% by weight to 90% by weight. The isoflavones genestin and daidzin comprise the major portion of isoflavones found in soy. Useful soy isoflavones include powdered soy isoflavones, an example of which is commercially available, e.g., under the trade designation NOVASOY (40% soy isoflavones) from Archer Daniels Midland Company (Decatur, Ill.). Preferably soy isoflavones are present in the composition in an amount sufficient to temporarily reduce hot flashes, decrease levels of breast cancer or a combination thereof. Preferably the composition includes no greater than 20% by weight, from about 1% by weight to about 10% by weight, or even from about 1% by weight to about 5% by weight soy isoflavones. A single dose tablet preferably includes at least about 5 mg, at least about 10 mg, at least about 20 mg, at least about 30 mg, no greater than about 100 mg, no greater than about 80 mg, or even no greater than 60 mg soy isoflavones.

The effervescent composition can optionally include a variety of additional active agents including N-acetyl-L-cysteine and ascorbic acid (i.e., vitamin C).

N-acetyl-L-cysteine has been reported to support the immune system, act as a free radical detector, and aid in prevention of bone loss. A suitable source of N-acetyl-L-cysteine is the N-acetyl-L-cysteine commercially available in powder form from NutriScience Innovations, LLC (Fairfield, Conn.). Preferably the composition includes no greater than 20% by weight, from about 3% by weight to about 20% by weight, or even from about 3% by weight to about 10% by weight N-acetyl-L-cysteine. A single dose tablet preferably includes at least about 50 mg, at least about 70 mg, at least about 80 mg, no greater than about 200 mg, no greater than about 150 mg, or even no greater than about 120 mg N-acetyl-L-cysteine.

Vitamin C has been reported to enhance the immune system, aid in osteoporosis prevention by preventing bone loss, and reduce hot flashes. Preferably the composition includes no greater than 10% by weight, from about 2% by weight to about 10% by weight, or even from about 2% by weight to about 5% by weight vitamin C. A single dose tablet preferably includes at least about 10 mg, at least about 25 mg, at least about 55 mg, no greater than about 150 mg, no greater than about 100 mg, or even no greater than about 80 mg vitamin C.

The effervescent agent is an effervescent couple that includes an acid and a base. The effervescent couple is activated when contacted with water, e.g., when the effervescent powder or tablet is placed in a glass of water. The water liberates the acid and base and enables the acid and base to react with each other to produce carbon dioxide gas, which imparts carbonation to the aqueous composition. Examples of useful acids include citric acid, ascorbic acid, aspartic acid, malic acid, adipic acid, tartaric acid, fumaric acid, succinic acid, sodium acid pyrophosophate, lactic acid, hexamic acid, amino acids, and acid salts and acid anhydrides thereof, and mixtures thereof. Examples of useful acid anhydrides include citraconic anhydride, glucono-D-lactone, and succinic anhydride. Examples of useful acid salts include potassium bitartrate, acid citrate salts, sodium dihydrogen phosphate, disodium dihydrogen phosphate, sodium acid sulfite, and combinations thereof. Preferably acid is present in the composition in an amount of from 10% by weight to about 60% by weight, from about 15% by weight to about 50% by weight, or even from about 25% by weight to about 40% by weight.

The base preferably is capable of generating carbon dioxide. Examples of suitable carbonate bases include sodium bicarbonate, sodium carbonate, sodium sesquicarbonate, potassium carbonate, potassium bicarbonate, calcium carbonate, magnesium carbonate, magnesium oxide, sodium glycine carbonate, L-lysine carbonate, arginine carbonate, zinc carbonate, zinc oxide, amino acid carbonates, and mixtures thereof. Selecting calcium carbonate has the beneficial health effect of adding calcium to the composition. Calcium has been reported to aid in osteoporosis prevention by preventing bone loss. The composition preferably includes base in an amount of from 10% by weight to about 60% by weight, from about 15% by weight to about 50% by weight, or even from about 25% by weight to about 40% by weight.

The composition can also include other ingredients including, e.g., flavor agents, fillers, surfactants (e.g., polysorbate 80 and sodium lauryl sulfate), color agents including, e.g., dyes and pigments, and sweeteners.

Useful flavor agents include natural and synthetic flavoring sources including, e.g., volatile oils, synthetic flavor oils, flavoring aromatics, oils, liquids, oleoresins and extracts derived from plants, leaves, flowers, fruits, stems and combinations thereof. Useful flavor agents include, e.g., citric oils, e.g., lemon, orange, grape, lime and grapefruit, fruit essences including, e.g., apple, pear, peach, grape, strawberry, raspberry, cherry, plum, pineapple, apricot, and other fruit flavors. Other useful flavor agents include, e.g., aldehydes and esters (e.g., benzaldehyde (cherry, almond)), citral, i.e., alpha-citral (lemon, lime), neral, i.e., beta-citral (lemon, lime), decanal (orange, lemon), aldehyde C-8 (citrus fruits), aldehyde C-9 (citrus fruits), aldehyde C-12 (citrus fruits), tolyl aldehyde (cherry, almond), 2,6-dimethyloctanal (green fruit), 2-dodedenal (citrus, mandarin) and mixtures thereof.

Useful color agents include, e.g., food, drug and cosmetic (FD&C) colors including, e.g., dyes, lakes, and certain natural and derived colorants. Useful lakes include dyes absorbed on aluminum hydroxide and other suitable carriers.

Useful sweetening agents include stevia, sugars such as sucrose, glucose, invert sugar, fructose, ribose, tagalose, sucralose, malitol, erythritol, xylitol, and mixtures thereof, saccharin and its various salts (e.g., sodium and calcium salt of saccharin), cyclamic acid and its various salts, dipeptide sweeteners (e.g., aspartame), acesulfame potassium, dihydrochalcone, glycyrrhizin, and sugar alcohols including, e.g., sorbitol, sorbitol syrup, mannitol and xylitol, and combinations thereof.

The effervescent composition can be in a variety of forms including, e.g., powder (e.g., a free flowing granulation), tablet, capsule, pellet and composite. When in the form of a tablet, the composition preferably includes binder, lubricant, and combinations thereof. Examples of suitable binders include, e.g., starches, natural gums, cellulose gums, microcrystalline cellulose, methylcellulose, cellulose ethers, sodium carboxymethylcellulose, ethylcellulose, gelatin, dextrose, lactose, sucrose, sorbitol, mannitol, polyethylene glycol, polyvinylpyrrolidone, pectins, alginates, polyacrylamides, polyvinyloxoazolidone, polyvinylalcohols and mixtures thereof.

When binder is present, the composition includes a sufficient amount of binder to assist in holding the components of the composition together in the form of a tablet. When present, the composition preferably includes from 10% by weight to about 60% by weight, from about 15% by weight to about 50% by weight, or even from about 25% by weight to about 40% by weight binder.

Various lubricants are suitable for use in the composition including water dispersible, water soluble, water insoluble lubricants and combinations thereof. Preferred lubricants are water soluble. Examples of useful water soluble lubricants include sodium benzoate, polyethylene glycol, L-leucine, adipic acid, and combinations thereof. The composition can also include water insoluble lubricants including, e.g., stearates (e.g., magnesium stearate, calcium stearate and zinc stearate), oils (e.g., mineral oil, hydrogenated and partially hydrogenated vegetable oils, and cotton seed oil) and combinations thereof. Other water insoluble lubricants include, e.g., animal fats, polyoxyethylene monostearate, talc, and combinations thereof.

The composition preferably includes a sufficient amount of lubricant to enable the composition to be formed into tablets and released from a high speed tableting press in the form of a tablet. When present, the composition preferably includes from 1% by weight to about 15% by weight, from about 1% by weight to about 12% by weight, from about 2% by weight to about 10% by weight, or even from about 3% by weight to about 8% by weight lubricant.

When the effervescent composition is in the form of a tablet, the tablet preferably has a weight from about 1.5 g to about 8 g, from about 2 g to about 6 g, or even from about 2.5 g to about 5 g.

The effervescent composition is preferably stored in a moisture-proof package including, e.g., sealed metal foil pouches, blister packs, and desiccant capped tubes. The composition can be administered by placing the composition in excess water, e.g., an eight ounce glass of tap water, to form an aqueous dispersion, which is then ingested. After addition of the effervescent composition to an aqueous liquid, the composition optionally can be stirred to facilitate dispersion in the aqueous liquid.

The effervescent composition also exhibits good stability. The effervescent agent of an effervescent composition can decompose over time and evolve a gas such as carbon dioxide, which can cause the packaging in which the effervescent composition is located to expand, which is observed as “puffing”. Packages of the effervescent compositions preferably are essentially free of puffing, or even free of puffing, after storage at room temperature for 24 hours, one month, for three months, for six months or even for one year.

The invention will now be described by way of the following example. All amounts are in grams unless otherwise indicated.

### EXAMPLE

Test Procedures

Test procedures used in the examples include the following.

Disintegration Time

An effervescent tablet is placed in excess water, approximately 200 ml, having a temperature of about 22° C., and the amount of time required to achieve 100% disintegration is measured.

Stability Test Method

Individually packaged effervescent tablets are stored at conditions of room temperature, 45° C. and 40° C. at 75% relative humidity and periodically observed for puffiness, i.e., visible expansion or puffing of the packaging relative to the packaged tablet immediately after manufacture. The packages are also observed for tablet movement within the package. If there is no puffing or only slight tablet movement, the tablet is deemed to be stable.

### Example 1

Effervescent tablets were prepared by first drying herbal extracts for 16 hours at 45° C., sieving the ingredients in a Number 12 sieve, and then combining the ingredients in the amounts (reported in grams (g)) set forth in Table 1 with mixing. The formulation was mixed for nine minutes and then transferred to a tablet press 21 mm tool to form tablets weighing from approximately 2.8 g to 2.9 g. The tablets are pressed to a hardness of at least five kilopounds.

A tablet was placed in approximately 200 ml of room temperature water and observed to dissolve in less than 2.7 minutes.

Individual tablets were sealed in air tight foil packages and stored at room temperature, 45° C., and 40° C. at 75% relative humidity for up to 30 days. The observations are reported in Table 2.

TABLE 1

| Description | grams (g) |
|---|---|
| Citric acid | 450 |
| Sorbitol instant | 350 |
| Sodium carbonate | 50 |
| Sodium bicarbonate | 200 |
| Potassium bicarbonate | 100 |
| Mineral oil | 9 |
| Polyethylene glycol | 22.5 |
| Sucralose | 12.5 |
| Flavoring Agents | 51.8 |
| N-acetyl-L-cysteine² | 51.5 |
| Ascorbic acid | 30.3 |
| Red clover extract³ | 26.3 |
| Black cohosh extract (2.5% triperpene glycosides)⁴ | 21 |
| Sage extract⁵ | 15.8 |
| Soy isoflavones⁶ | 37.5 |
| Total | 1428.2 |

¹Trade designation VELTOL ULTRA 3100P manufactured by Danisco Belgium S.A. (Louvain-La-Neuve, Belgium).
²N-acetyl-L-cysteine USP 23 manufactured by NutriScience Innovations, LLC (Fairfield, Connecticut).
³Red Clover Powder manufactured by Buckton Scott Nutrition, Inc. (Fairfield, New Jersey).
⁴Black Cohosh P.E. 2.5% Triperpene Glycosides manufactured by BI Nutraceuticals (Long Beach, California).
⁵Sage Powder manufactured by Buckton Scott Nutrition, Inc. (Fairfield, New Jersey).
⁶Trade designation NOVASOY isoflavone compound (40% Isoflavones) manufactured by Archer Daniels Midland Company (Decatur, Illinois).

TABLE 2

| | | Observations: | Observations: |
|---|---|---|---|
| Stability Testing | Days of | Package | Tablet |
| Conditions | Storage | Puffing | Movement |
| 45° C. | 1 | None | None |
| 45° C. | 5 | None | Slight |
| 45° C. | 7 | None | Slight |
| 40° C./75% RH | 1 | None | None |
| 40° C./75% RH | 5 | None | Slight |
| 40° C./75% RH | 7 | None | Slight |
| 40° C./75% RH | 30 | None | Slight |
| Room Temperature | 1 | None | None |
| (about 25° C.) | | | |
| Room Temperature | 5 | None | None |
| (about 25° C.) | | | |
| Room Temperature | 7 | None | None |
| (about 25° C.) | | | |
| Room Temperature | 30 | None | None |
| (about 25° C.) | | | |

RH = Relative Humidity

Other embodiments are within the claims.

What is claimed is:

1. An effervescent composition comprising:
at least 0.5% by weight of herbal extract selected from the group consisting of red clover extract, black cohosh extract, sage extract, and combinations thereof;
at least about 0.1% by weight soy isoflavones;
at least about 0.1% by weight N-acetyl-L-cysteine; and
effervescent agent comprising acid and base.

2. The effervescent composition of claim 1, comprising at least 1.0% by weight said herbal extract.

3. The effervescent composition of claim 1 further comprising ascorbic acid.

4. The effervescent composition of claim 1 further comprising flavor agent, color agent, sweetener, or a combination thereof.

5. The effervescent composition of claim 1, wherein said acid comprises citric acid, tartaric acid, or a combination thereof.

6. An effervescent tablet comprising the composition of claim 1, said composition further comprising
binder; and
lubricant.

7. The effervescent tablet of claim 6, wherein said binder comprises sorbitol.

8. The effervescent tablet of claim 6, wherein said lubricant comprises at least one of mineral oil and polyethylene glycol.

9. The effervescent tablet of claim 6, said tablet exhibiting a hardness of at least 5 kilopounds and disintegrating in water having a temperature of about 22° C. in less than 3.5 minutes.

10. The effervescent tablet of claim 6, wherein said tablet, when stored in an airtight sealed package at 40° C. and 75% relative humidity for 7 days, is free of puffing.

11. The effervescent tablet of claim 6, wherein said tablet, when stored in an airtight sealed package at 40° C. and 75% relative humidity for 30 days, is free of puffing.

12. The effervescent tablet of claim 6, wherein said tablet comprises from about 20 mg to about 200 mg of said herbal extract.

13. The effervescent tablet of claim 6, wherein said tablet comprises from about 50 mg to about 100 mg soy isoflavones.

14. The effervescent tablet of claim 6, wherein said tablet comprises from about 70 mg to about 150 mg N-acetyl-L-cysteine.

15. The effervescent tablet of claim 6 further comprising from about 25 mg to about 100 mg ascorbic acid.

16. The effervescent tablet of claim 6, said tablet comprising
at least 30 mg black cohosh extract;
at least 30 mg red clover extract; and
at least 20 mg soy isoflavones.

17. The effervescent composition of claim 1, comprising no greater than about 20% by weight said soy isoflavones and no greater than about 20% by weight said N-acetyl-L-cysteine.

18. An effervescent composition comprising:
at least 30 mg of a first herbal extract selected from the group consisting of red clover extract, black cohosh extract, sage extract, and combinations thereof;
at least 30 mg of a second herbal extract selected from the group consisting of red clover extract, black cohosh extract, sage extract, and combinations thereof, said second herbal extract being different from said first herbal extract; and
effervescent agent comprising acid and base.

19. An effervescent composition comprising:
from about 1% by weight to about 5% by weight of soy isoflavones;
from about 1% by weight to about 5% by weight of red clover extract;
from about 1% by weight to about 5% by weight of black cohosh extract;
from about 1% by weight to about 5% by weight of sage extract;
from about 3% by weight to about 10% by weight of N-acetyl-L-cysteine; and
effervescent agent comprising acid and base.

20. A method of making the effervescent composition of claim 1, said method comprising
drying the herbal extract; and
combining said herbal extract with the effervescent agent.

21. A method of making the effervescent tablet of claim 6, said method comprising
drying the herbal extract;
combining said herbal extract with the effervescent agent; and
forming the effervescent composition into a tablet having a hardness of at least 3 kilopounds.
